(19) United States
(12) Patent Application Publication
(10) Pub. No.: US 2005/0154325 A1
(43) Pub. Date: Jul. 14, 2005

(54) Sudden cardiac arrest monitoring system

(75) Inventors: Josef Lauter, Geilenkirchen (DE); Christian Reichinger, Neutraubling (DE); Harald Reiter, Aachen (DE); Ralf Schmidt, Aachen (DE); Olaf Such, Aachen (DE); Gereon Vogtmeier, Aachen (DE)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics, Eindhoven (NL)

(21) Appl. No.: 10/509,416

(22) PCT Filed: Mar. 6, 2003
(86) PCT No.: PCT/IB03/00841

(51) Int. Cl. A61B 5/04

(52) U.S. Cl. 600/515; 128/903

(57) ABSTRACT

A wearable heart monitoring system (1) for monitoring of a cardiac arrhythmia, said system comprising ECG sensors (3) for providing patient heart data, a conditioning and interpreting circuitry (4) for processing the heart data, alarm generation means for generating an alarm, said conditioning and interpreting circuitry comprises a real-time evaluator for measuring and analyzing a histogram of a temporal distribution of an interval between successive corresponding characteristic peaks in an ECG spectrum during a plurality of successive heart cycles, the alarm generation means being arranged to generate an alarm based on the analysis of said histogram.

The invention relates to a wearable heart monitoring system for monitoring of a cardiac arrhythmia, said system comprising ECG sensors for providing patient heart data, a conditioning and interpreting circuitry for processing the heart data, alarm generation means for generating an alarm.

The invention further relates to a method for alerting a patient for an substantial probability of a cardiac arrest event, said method being based on results of continuous monitoring of a cardiac activity by means of a cardiac monitoring system comprising a set of electrodes, a conditioning and interpreting circuitry and alarm generation means.

A system of a kind described above is known from U.S. Pat. No. 5,634,468. The known system is a wearable monitoring system, which is used by the patients outside a hospital environment. The known system comprises a sensor patch for performing an ECG monitoring, a conditioning and interpreting circuitry for performing an analysis of actual cardiac cycles of the patient and comparing said actual cardiac cycle with a template cycle for drawing a conclusion about an actual condition of the patient. In case an abnormal condition of the patient is detected by the system, an alarm is generated for alarming the medical staff located at a remote position.

The disadvantage of the known system is that this system is poorly suited for a sudden cardiac arrest monitoring as a response time for the medical personnel must fall within few minutes, which is difficult to realize.

It is an object of the invention to provide a wearable monitoring system where a possible sudden cardiac arrest is sufficiently anticipated to provide a broader temporal margin for a medical response.

For this purpose the wearable heart monitoring system is characterized in that said conditioning and interpreting circuitry comprises a real-time evaluator for measuring and analyzing a histogram of a temporal distribution of an interval between successive corresponding characteristic peaks in an ECG spectrum during a plurality of successive heart cycles, the alarm generation means are being arranged to generate an alarm based on the analysis of said histogram. This technical measure is based on an insight that prior to the event of sudden cardiac arrest some patients are experiencing an arrythmia for a prolonged period of time, the arrythmia condition being characterized by a specific temporal distribution of a characteristic interval in the ECG spectrum. For these persons it is therefore sufficient to analyze the interval between corresponding peaks of a ECG spectrum and in case this interval repetitively shifts to a shorter time intervals to generate an alarm. In such cases the patient will have enough time before sudden cardiac arrest occurs to go to the hospital, for example. Then an expensive ICD (internal cardiac defibrillator) therapy could provide a greater probability for a person to survive a sudden cardiac arrest. An R-peak of the ECG-spectrum is a well-suited peak for such an analysis. It must be noted, that it is also possible to use other peaks for this purpose.

An embodiment of the system according to the invention is characterized in that said system further comprises an RF-link for transmitting a further alarm to a remote monitoring station. This particular technical measure is advantageous in order to alert the medical personnel at the remote location, such as an emergency calling center or a hospital's first aid department, to inform the personnel about the patient exhibiting heart problems which could lead to a sudden cardiac arrest. It must be noted that in the system according to the invention only the alarm is transferred by means of an RF-link. All necessary data manipulation and analysis, such as a calculation of the histogram of the temporal distribution of an interval between successive corresponding characteristic peaks and a comparison thereof with a normal condition, are performed at the patient's site by means of the conditioning and interpreting circuitry. Such a functionality ensures a low power consumption by the monitoring system contributing to its durability.

A further embodiment of the system is characterized in that the ECG sensors are housed on an elastic belt. It has been established that in order to analyze the patient's condition based upon the interval between the successive corresponding peaks in the ECG, it is possible to locate the sensors at the thorax region or at the abdominal region. By integrating of the ECG sensors in a clothing, for example a belt of an underwear slip of a brassier a patient-friendly monitoring system can be obtained. By means of the elastic belt the sensors are constantly put under the necessary pressure to ensure a constant position of the sensors with respect to the patient's skin. In case the wiring is integrated in the fabric of the elastic belt as well, a monitoring system can be obtained providing a maximum convenience and privacy to the recipient. An example of a suitable electrode material is a per se known electrically conductive rubber which has a certain degree of stretchability as well adding to the patient's comfort. By sealing off the electrical contacts between the electrode material and the wiring a washable wearable monitoring system can be provided.

A still further embodiment of the monitoring system according to the invention is characterized in that said system further comprises a motion sensor. It is advantageous to provide a motion detector together with the monitoring system according to the invention. The motion detector can be arranged to prevent the monitoring system from gathering false data in case of a too extensive body movement.

The method for alerting a patient for an substantial probability of a cardiac arrest event according to the invention is characterized in that said method comprises the steps of performing a continuous acquisition of data related to the cardiac activity by means of the electrodes; processing the data for extracting a characteristic parameter by means of the conditioning and interpreting circuitry; performing a classification of the extracted characteristic parameter; generating an alarm with alarm means in case the characteristic parameters falls within an alarm-relevant category.

These and other aspects of the invention will be further elaborated with reference to figures.

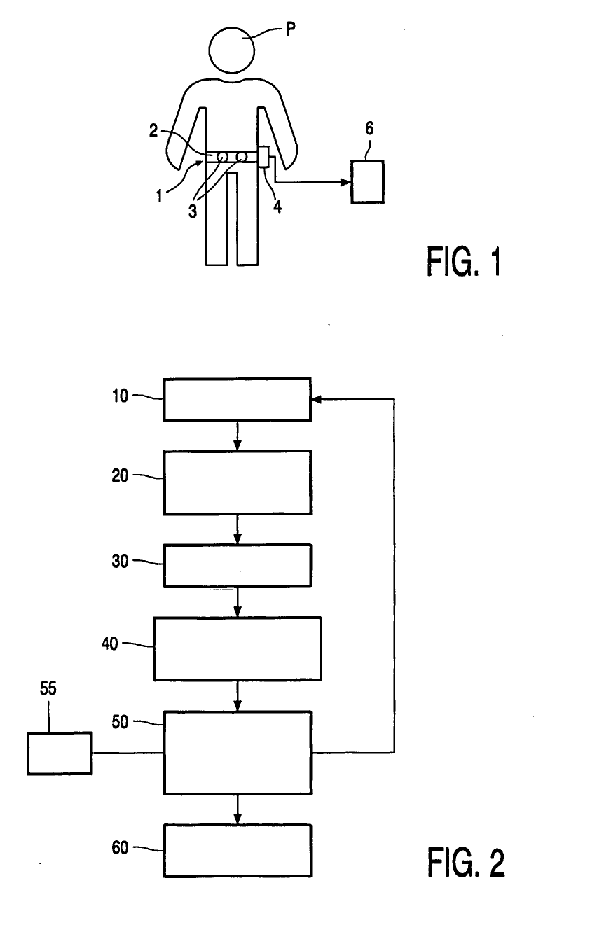

FIG. 1 shows schematically an embodiment of a wearable heart monitoring system.

FIG. 2 shows schematically an embodiment of a conditioning and interpreting circuitry.

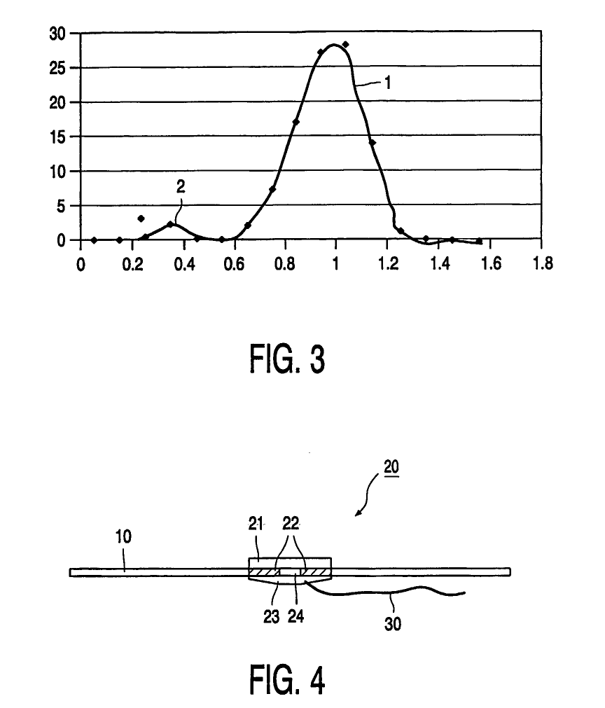

FIG. 3 shows schematically an example of a R-R interval distribution corresponding to the arrythmia condition.

FIG. 4 shows schematically an embodiment of the monitoring system according to the invention being integrated into a garment.

FIG. 1 presents a schematic view of an embodiment of the wearable heart monitoring system 1 according to the invention. A patient P is supplied with a set of sensors 3, for example integrated on an elastic belt 2. The set of sensors 3 are thus reliably positioned in a contact with the patient's skin in order to acquire a desirable physiological signal. For example, such a monitoring system can be arranged to perform measurements of the heart activity or a signal derived from the heart activity. The sensors 3 comprising electrodes (not shown) are electrically connected to the storage and analysis device (SAD) 4. The SAD is arranged to perform a primary data analysis in order to interpret the acquired physiological data. The data analysis is being performed by means of a conditioning and interpreting circuitry, further elaborated in FIG. 2. In case the conditioning and interpreting circuitry detects an life threatening abnormality (for example an arrythmia condition or a cardiac arrest) a control signal, for example, an alarm is being sent to a remote station 6. The remote station 6 can be located in a nearby hospital or at a emergency call center specializing on first aid for cardiac arrest conditions. It is found that in order to detect the event of the cardiac arrest it is sufficient to use only two sensors which can be integrated in the belt. Next to these sensors a motion detector can be assembled on the elastic belt. Any portable motion detector known in the art is suitable for this purpose. In a case of the cardiac arrest event the patient is not moving, which is conferred by the motion sensor. In this case (no patient movement) it is found to be feasible to measure a full ECG with two sensors localized in the abdominal area of the patient [correct?].

FIG. 2 shows schematically an embodiment of a conditioning and interpreting circuitry which enables to trigger an alarm when a cardiac arrest is anticipated, but not yet has occurred. The conditioning and interpreting circuitry analyses a raw body signal 10 acquired by means of electrodes. Such a raw body signal can be a heart cycle comprising P,Q,R,S,T characteristic peaks. The raw body signal 10 is subsequently submitted to a baseline and interference filter 20 in order to subtract signal interferences. The filtered signal is then subjected to an interval detector 30, where a temporal interval between the subsequent characteristic peaks of one type is calculated. For example, an R-peak is a suitable characteristic peak for this purpose. However, other characteristic peaks, such as P,Q,S,T can be used for this purpose as well. After the interval has been determined it is submitted to a real-time evaluator 40, where the value of the interval is being classified and stored. An example of such a classification is a temporal histogram. After the interval is classified in the real-time evaluator 40, a pre-stored look-up table, schematically indicated by 55, is inquired by the logical unit 50 in order to condition whether an alarm-relevant classification has occurred. An alarm-relevant classification can be in case of an anythmia or, when the arrythmia did not occur, in case of a cardiac arrest. In both cases an alarm is generated by the alarm means 60, preferably with an alarm of a high priority in case of the cardiac arrest. In case the detected event is not classified as an abnormality the system sends a signal to the sub-unit which continues with analyzing a following sample of the raw patient data 10. It has been understood that such a monitoring system is advantageous, as it gives the possibility to recognize an approaching cardiac arrest by means of the arrythmia detection. In this case the patient has a broader temporal margin to seek for help or an alarmed medical personnel has a broader temporal margin for assistance. Both lead to a higher probability of survival in case of an approaching cardiac arrest. Next to this due to the fact that the system electronics measures only the intervals in the heart cycle and stores the distribution of the intervals it has a low power consumption leading to an increased durability of the monitoring system. A change in the interval distribution is used as an indicator of an abnormality. In order to decrease false alarms the system can be further supplied with a threshold value for a selected characteristic peak interval, above which no alarm is being generated. It is further possible to arrange the system to be a self-learning one, iteratively increasing the threshold value in case repetitive false alarms are being generated. Furtheron, in certain diagnostic intervals (each day, each week, etc) the distribution can be transferred with the wireless connection or with a plug connection to a remote station for diagnostic purposes.

FIG. 3 shows schematically an example of a R-R interval distribution corresponding to the arrythmia condition. The peak 1 corresponds to a normal heart activity of the patient. The upcoming peak 2 is a characteristic distribution in case of arrythmia. Therefore, the upcoming peak at shorter R-R intervals can be used as an indicator for possible future cardiac arrest. The peak 2 occurs at the R-peak interval value of 0.35 s, corresponding to arrythmia. This value depends on the duration of the time frame of the interpretation. The trigger level for an alarm can be chosen according to the duration of the time window for the observation.

FIG. 4 shows schematically an embodiment of a part of the monitoring system according to the invention being integrated into a garment. Electrodes 20 [only one electrode is being shown] to acquire patient data are attached to the elastic belt 10. The electrode material 20 can be manufactured from a mixture of electrically conductive graphite with a silicon gel, for example. Other suitable materials can also be used to manufacture the electrode, for example conductive rubbers. The electrode body can be attached to the elastic belt 10 by any of the procedures: molding, gluing or knitting, etc. It is also possible that the elastic belt is pre-processed to comprise cut-aways in areas where the electrodes are to be positioned. As is shown in FIG. 4, a part of the electrode, indicated by 24, is located in such a cut-away of the belt 10 and another part of the electrode, indicated by 22, is joined with the elastic belt 10. The outer body of the electrode can be covered by the mould (not shown) at the backside surface 23 of the electrode 20. The electrical connections to the electrode 20 are realized by means of a wire, schematically presented by 30. This wire 30 leads to other electrodes in the monitoring system and to the motion sensor (optional) and to the Storage and Analysis Device (not shown) also attached to the belt. Thus, a cheap, durable and reliable wearable monitoring system can easily be realized using the method of the invention.

1. A wearable heart monitoring system for monitoring of a cardiac arrhythmia, said system comprising ECG sensors for providing patient heart data, a conditioning and interpreting circuitry for processing the heart data, alarm generation means for generating an alarm, characterized in that said conditioning and interpreting circuitry comprises a real-time evaluator for measuring and analyzing a histogram of a temporal distribution of an interval between successive corresponding characteristic peaks in an ECG spectrum during a plurality of successive heart cycles, the alarm generation means being arranged to generate an alarm based on the analysis of said histogram.

2. A system according to claim 1, characterized in that said system further comprises an RF-link for transmitting a further alarm to a remote monitoring station.

3. A system according to claim 1, characterized in that the ECG sensors are housed on an elastic belt.

4. A system according to claim 3 characterized in that the system comprises electrical wiring for arranging electrical connections of the monitoring system, said wiring being integrated in the belt.

5. A system according to claim 4, characterized in that a wire material has a substantially the same elasticity as a material constituting the elastic belt.

6. A system according to claim 5, characterized in that said system comprises at least two electrodes.

7. A monitoring system according to one of claim 1, characterized in that said system further comprises a motion sensor.

8. A method for alerting a patient for an substantial probability of a cardiac arrest event, said method being based on results of continuous monitoring of a cardiac activity by means of a cardiac monitoring system comprising a set of electrodes, a conditioning and interpreting circuitry and alarm generation means, characterized in that said method comprises the steps of:
performing a continuous acquisition of data related to the cardiac activity by means of the electrodes;
processing the data for extracting a characteristic parameter by means of the conditioning and interpreting circuitry;
performing a classification of the extracted characteristic parameter;
generating an alarm with alarm means in case the characteristic parameters falls within an alarm-relevant category.

9. A method for alerting a patient for an substantial probability of a cardiac arrest event according to claim 8, characterized in, that an alarm with a high priority is generated in case of a sudden cardiac arrest.
